(19) United States
(12) Patent Application Publication
(10) Pub. No.: US 2023/0234908 A1
(43) Pub. Date: Jul. 27, 2023

(54) REMOVAL OF ALDEHYDES IN ACETIC ACID PRODUCTION

(71) Applicant: LyondellBasell Acetyls, LLC, Houston, TX (US)

(72) Inventors: Noel C. Hallinan, Loveland, TX (US); David L. Ramage, Friendswood, TX (US); Brian A. Salisbury, Beach City, TX (US)

(73) Assignee: LyondellBasell Acetyls, LLC, Houston, TX (US)

(21) Appl. No.: 18/095,099

(22) Filed: Jan. 10, 2023

Related U.S. Application Data
(60) Provisional application No. 63/298,437, filed on Jan. 11, 2022.

(51) Int. Cl. C07C 51/47; C07C 51/44

(52) U.S. Cl. CPC C07C 51/47; C07C 51/44

(57) ABSTRACT

A system and method for removing acetaldehyde from an acetic acid system, including providing a solution from the acetic acid system, the stream having methyl iodide and acetaldehyde, and contacting the solution with a polymer-bound polyol.

### PRIOR RELATED APPLICATIONS

This application claims the benefit of priority to U.S. Provisional Application No. 63/298,437, filed on Jan. 11, 2022, the contents of which are incorporated herein by reference in their entirety.

### FIELD OF THE INVENTION

This disclosure relates to the production of acetic acid. More particularly, the disclosure relates to removal of acetaldehyde in acetic acid production.

### BACKGROUND OF THE INVENTION

In the current acetic acid production process, a reaction mixture is withdrawn from a reactor and is separated in a flash tank into a liquid fraction and a vapor fraction comprising acetic acid generated during the carbonylation reaction. The liquid fraction may be recycled to the carbonylation reactor, and the vapor fraction is passed to a separations unit, which by way of example may be a light-ends distillation column. The light-ends distillation column separates a crude acetic acid product from other components. The crude acetic acid product is passed to a drying column to remove water and then is subjected to further separations to recover acetic acid.

One challenge facing the industry is the presence of aldehyde(s) in acetic acid production, which can be present in the feed and also form as an undesired byproduct of carbonylation reactions. Processes for removing aldehydes exist; however, there continues to be a need to improve upon, and provide alternatives to, current aldehyde removal processes.

### SUMMARY OF THE INVENTION

An aspect of the disclosure relates to a method for removing acetaldehyde from an acetic acid system, including: providing a solution from the acetic acid system, the solution comprising methyl iodide and acetaldehyde; and contacting the solution with a polymer-bound polyol resin to reduce the acetaldehyde content of the solution.

Another aspect of the disclosure relates to a method of operating an acetic acid production system, including: flashing a reaction mixture discharged from an acetic acid production reactor into a vapor stream and a liquid stream, the vapor stream comprising acetic acid, water, methanol, methyl acetate, methyl iodide, and acetaldehyde; distilling the vapor stream into a product stream of acetic acid and water, a bottoms stream, and an overhead stream comprising methyl iodide, water, methyl acetate, acetic acid, and acetaldehyde; condensing the overhead stream into a light aqueous phase comprising water, acetic acid, and methyl acetate, and a heavy organic phase comprising methyl iodide, acetic acid, water, and a first concentration of acetaldehyde; and contacting at least a portion of the heavy organic phase with a polymer-bound polyol, wherein the polymer-bound polyol adsorbs a portion of the acetaldehyde to produce a treated heavy organic phase, wherein the treated heavy organic phase has a second concentration of acetaldehyde, and the second concentration is lower than the first concentration. Without wishing to be bound by any theory, it is believed that adsorption occurs as a result of formation of bound acetals.

Yet another aspect relates to a method of producing acetic acid, including: reacting methanol and carbon monoxide in the presence of a carbonylation catalyst to produce a crude stream comprising acetic acid; purifying the crude stream to produce a product stream comprising the acetic acid, wherein the purifying generates a methyl iodide stream comprising methyl iodide and acetaldehyde; and contacting at least a portion of the methyl iodide stream with a heavy organic phase with a polymer-bound polyol to adsorb a portion of the acetaldehyde to reduce an amount of acetaldehyde in an acetic acid system producing the acetic acid.

Yet another aspect of the disclosure relates to an acetic acid production system, having: a reactor to react methanol and carbon monoxide in the presence of a carbonylation catalyst to form acetic acid; a flash vessel that receives a reaction mixture comprising the acetic acid from the reactor; a distillation column that receives a vapor stream from the flash vessel; a decanter that receives a condensed overhead stream from the distillation column; and a flow-through bed that receives a heavy organic phase from the decanter, wherein the heavy organic phase comprises methyl iodide and acetaldehyde, and the flow-through bed comprises a polymer-bound polyol to adsorb acetaldehyde.

The above paragraphs present a simplified summary of the presently disclosed subject matter in order to provide a basic understanding of some aspects thereof. The summary is not an exhaustive overview, nor is it intended to identify key or critical elements to delineate the scope of the subject matter claimed below. Its sole purpose is to present some concepts in a simplified form as a prelude to the more detailed description set forth below.

### BRIEF DESCRIPTION OF THE DRAWINGS

The claimed subject matter may be understood by reference to the following description taken in conjunction with the accompanying drawings, in which like reference numerals identify like elements, and in which:

FIG. 1 is a schematic of an exemplary acetic acid production system in accordance with embodiments of the present techniques;

FIG. 1A is a schematic of an exemplary continuation of FIG. 1 in accordance with embodiments of the present techniques;

FIG. 2 is an overlaid graph of % acetaldehyde (“HAc”), crotonaldehyde, and paraldehyde versus time for a solution of HAc with a polymer-bound polyol in accordance with embodiments of the present techniques;

FIG. 3 is an overlaid graph of % HAc, crotonaldehyde, and paraldehyde versus time for a solution of HAc with a polymer-bound polyol and an acid catalyst resin for comparison to embodiments of the present techniques;

FIG. 4 is an overlaid graph of % HAc, crotonaldehyde, and paraldehyde versus time for a solution of HAc with an acid catalyst resin for comparison to embodiments of the present techniques;

FIG. 5 is an overlaid graph of % HAc, crotonaldehyde, and paraldehyde versus time for a solution of HAc with a polymer-bound polyol and hydrogen iodide (“HI”) solution for comparison to embodiments of the present techniques; and

FIG. 6 is a graph of % HAc remaining in solution after contacting a polymer-bound polyol resin flow-through bed at various flow rates;

While the disclosed process and system are susceptible to various modifications and alternative forms, the drawings illustrate specific embodiments herein described in detail by way of example. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

A detailed description of embodiments of the disclosed process follows. However, it is to be understood that the described embodiments are merely exemplary of the process and that the process may be embodied in various and alternative forms of the described embodiments. Therefore, specific procedural, structural and functional details which are addressed in the embodiments described herein are not to be interpreted as limiting, but merely as a basis for the claims and as a representative basis for teaching one skilled in the art to variously employ the disclosed process.

The designation of groups of the Periodic Table of the Elements as used herein is in accordance with the current IUPAC convention. The expression “HAc” is used herein as an abbreviation for acetaldehyde. The expression “HI” is used herein as an abbreviation for hydrogen iodide. The expression “acac” is used herein as an abbreviation for acetoacetate anion, i.e., H₃CC(=O)CH₂C(=O)O-. Unless specifically indicated otherwise, the expression “wt%” as used herein refers to the percentage by weight of a particular component in the referenced composition. With respect to all ranges disclosed herein, such ranges are intended to include any combination of the mentioned upper and lower limits even if the particular combination is not specifically listed.

Embodiments of the disclosed process and system involve the production of acetic acid by carbonylating methanol in a carbonylation reaction. The carbonylation reaction may be represented by: CH₃OH+CO➛CH₃COOH

Embodiments of the disclosed process include: (a) obtaining HI in an acetic acid production system; and (b) continuously introducing a complexing agent into the system, wherein the complexing agent and HI interact to form a complex. The following description elaborates upon the disclosed process.

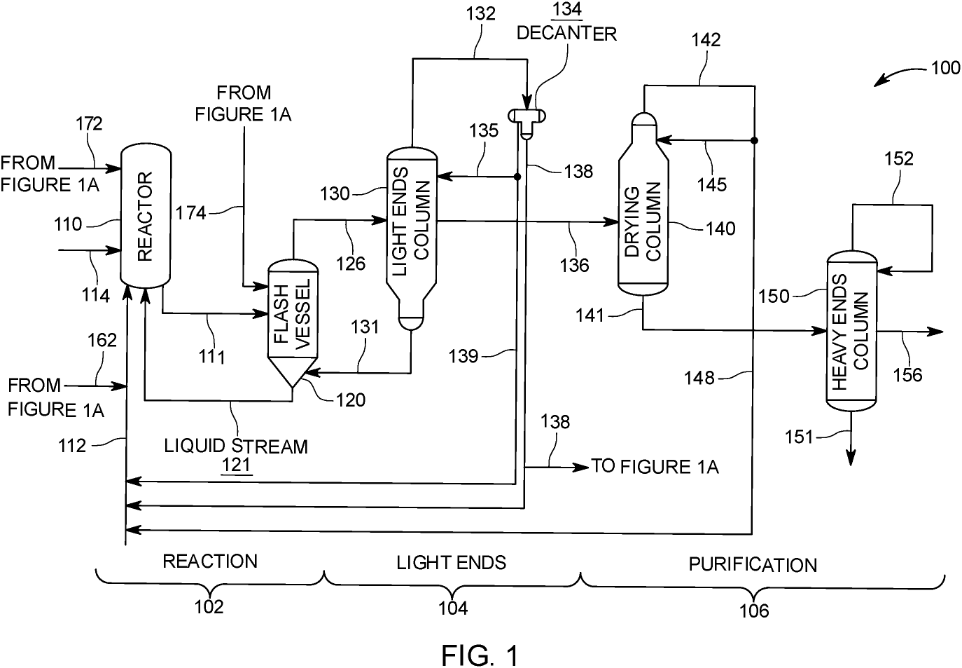

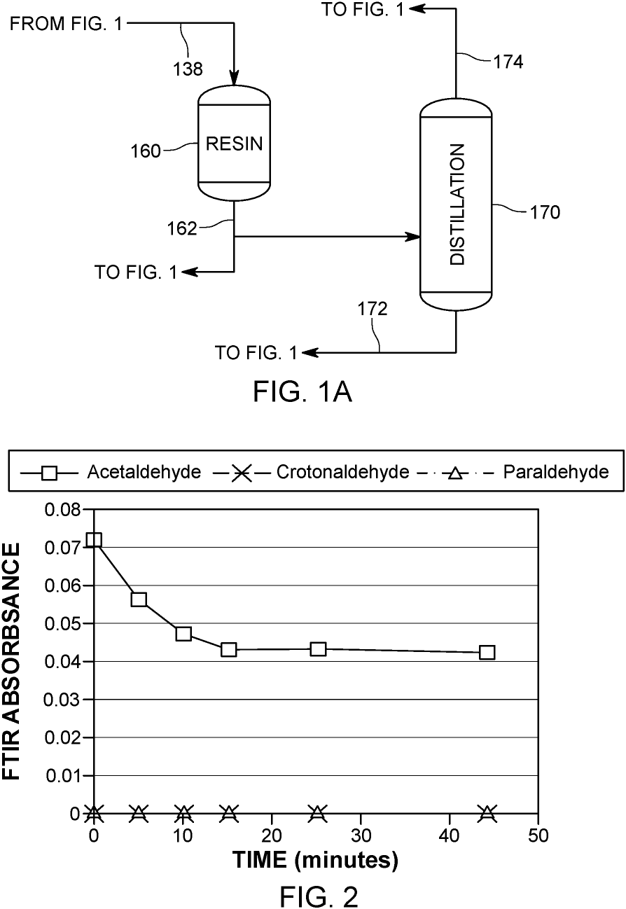

FIG. 1 is a schematic of an exemplary acetic acid production system 100 implementing the carbonylation reaction. In certain embodiments, the acetic acid system 100 may include a reaction area 102, a light-ends area 104, and a purification area 106. The reaction area 102 may include a reactor 110, a flash vessel 120, and associated equipment. The reactor 110 is a reactor or vessel in which methanol is carbonylated in the presence of a catalyst to form acetic acid at elevated pressure and temperature.

The flash vessel 120 is a tank or vessel in which a reaction mixture obtained in the reactor is at least partially depressurized and/or cooled to form a vapor stream and a liquid stream. The liquid stream 121 may be a product or composition which has components in the liquid state under the conditions of the processing step in which the stream is formed. The vapor stream 126 may be a product or composition which has components in the gaseous state under the conditions of the processing step in which the stream is formed.

The light-ends area 104 may include a separations column, for example a light-ends column 130, and associated equipment such as decanter 134. The light-ends column is a fractioning or distillation column and includes equipment associated with the column, such as heat exchangers, decanters, pumps, compressors, valves, and the like. The purification area 106 may include a drying column 140, optionally a heavy-ends column 150, and associated equipment, and so on. The heavy-ends column is a fractioning or distillation column and includes any equipment associated with the column, such as heat exchangers, decanters, pumps, compressors, valves, and the like. Further, as discussed below, various recycle streams may include streams 121, 138, 139, and 148. The recycle streams may be products or compositions recovered from a processing step downstream of the flash vessel 120 and which is recycled to the reactor 110, flash vessel 120, or light-ends column 130, and so forth.

In an embodiment, the reactor 110 may be configured to receive a carbon monoxide feed stream 114 and a methanol feed stream 112. A reaction mixture may be withdrawn from the reactor in stream 111. Other streams may be included such as, for example, a stream that may recycle a bottoms mixture of the reactor 110 back into the reactor 110, or a stream may be included to release a gas from the reactor 110.

In an embodiment, the flash vessel 120 may be configured to receive stream 111 from the reactor 110. In the flash vessel 120, stream 111 may be separated into a vapor stream 126 and a liquid stream 121. The vapor stream 126 may be communicated to the light-ends column 130, and the liquid stream 121 may be communicated to the reactor 110. In an embodiment, stream 126 may have acetic acid, water, methyl iodide, methyl acetate, HI, mixtures thereof and the like.

In an embodiment, the light-ends column 130 may be a distillation column and associated equipment such as a decanter 134, pumps, compressors, valves, and other related equipment. The light-ends column 130 may be configured to receive stream 126 from the flash vessel 120. In the illustrated embodiment, stream 132 is the overhead product from the light-ends column 130, and stream 131 is bottoms product from the light-ends column 130. As indicated, light-ends column 130 may include a decanter 134, and stream 132 may pass into decanter 134.

Stream 135 may emit from decanter 134 and recycle back to the light-ends column 130. Stream 138 may emit from decanter 134 and may recycle back to the reactor 110 via, for example, stream 112 or be combined with any of the other streams that feed the reactor. Stream 139 may recycle a portion of the light phase of decanter 134 back to the reactor 110 via, for example, stream 112. Stream 136 may emit from the light-ends column 130. Other streams may be included such as, for example, a stream that may recycle a bottoms mixture of the light-ends column 130 back into the light-ends column 130. Streams received by or emitted from the light-ends column 130 may pass through a pump, compressor, heat exchanger, and the like as is common in the art.

In an embodiment, the drying column 140 may be a vessel and associated equipment such as heat exchangers, decanters, pumps, compressors, valves, and the like. The drying column 140 may be configured to receive stream 136 from the light-ends column 130. The drying column 140 may separate components of stream 136 into streams 142 and 141. Stream 142 may emit from the drying column 140, recycle back to the drying column via stream 145, and/or recycle back to the reactor 110 through stream 148 (via, for example, stream 112). Stream 141 may emit from the drying column 140 and may include de-watered crude acetic acid product. Stream 142 may pass through equipment such as, for example, a heat exchanger or separation vessel before streams 145 or 148 recycle components of stream 142. Other streams may be included such as, for example, a stream may recycle a bottoms mixture of the drying column 140 back into the drying column 140. Streams received by or emitted from the drying column 140 may pass through a pump, compressor, heat exchanger, separation vessel, and the like as is common in the art.

The heavy-ends column 150 may be a distillation column and associated equipment such as heat exchangers, decanters, pumps, compressors, valves, and the like. The heavy-ends column 150 may be configured to receive stream 141 from the drying column 140. The heavy-ends column 150 may separate components from stream 141 into streams 151, 152, and 156. Streams 151 and 152 may be sent to additional processing equipment (not shown) for further processing. Stream 152 may also be recycled, for example, to light-ends column 140. Stream 156 may have acetic acid product.

A single column (not depicted) may be used in the place of the combination of the light-ends distillation column 130 and the drying column 140. The single column may vary in the diameter/height ratio and the number of stages according to the composition of vapor stream from the flash separation and the requisite product quality. For instance, U.S. Pat. No. 5,416,237, the teachings of which are incorporated herein by reference, discloses a single column distillation. Alternative embodiments for the acetic acid production system 100 may also be found in U.S. Pat. Nos. 6,552,221, 7,524,988, and 8,076,512, which are herein incorporated by reference.

In an embodiment, the carbonylation reaction in reactor 110 of system 100 may be performed in the presence of a catalyst. Catalysts may include, for example, rhodium catalysts and iridium catalysts.

Suitable rhodium catalysts are taught, for example, by U.S. Pat. No. 5,817,869, which is herein incorporated by reference. The rhodium catalysts may include rhodium metal and rhodium compounds. In an embodiment, the rhodium compounds may be selected from the group consisting of rhodium salts, rhodium oxides, rhodium acetates, organo-rhodium compounds, coordination compounds of rhodium, the like, and mixtures thereof in an embodiment, the rhodium compounds may be selected from the group consisting of Rh₂(CO)₄I₂, Rh₂(CO)₄Br₂, Rh₂(CO)₄Cl₂, Rh(CH₃CO₂)₂, Rh(CH₃CO₂)₃, [H]Rh(CO)₂I₂, the like, and mixtures thereof. In an embodiment, the rhodium compounds may be selected from the group consisting of [H]Rh(CO)₂I₂, Rh(CH₃CO₂)₂, the like, and mixtures thereof.

Suitable iridium catalysts are taught, for example, by U.S. Pat. No. 5,932,764. The iridium catalysts may include iridium metal and iridium compounds. Examples of suitable iridium compounds include IrCl3, IrI3, IrBr₃, [Ir(CO)₂I]₂, [Ir(CO)₂Cl]₂, [Ir(CO)₂Br]₂, [Ir(CO)4I₂]-H+, [Ir(CO)₂Br_{2]}-H+, [IR(CO)₂I₂]-H+, [Ir(CH₃)I₃(CO)₂]-H+, Ir4(CO)12, IrCl₃. 4H₂O, IrBr₃. 4H₂O, Ir₃(CO)1₂, Ir₂O3, IrO₂, Ir(acac)(CO)₂, Ir(acac)₃, Ir(OAc)₃, [Ir₃O(OAc)₆(H₂O)₃][OAc], H2[IrCl₆], the like, and mixtures thereof. In an embodiment, the iridium compounds may be selected from the group consisting of acetates, oxalates, acetoacetates, the like, and mixtures thereof. In an embodiment, the iridium compounds may be one or more acetates.

In an embodiment, the catalyst may be used with a co-catalyst. In an embodiment, co-catalysts may include metals and metal compounds selected from the group consisting of osmium, rhenium, ruthenium, cadmium, mercury, zinc, gallium, indium, and tungsten, their compounds, the like, and mixtures thereof. In an embodiment, co-catalysts may be selected from the group consisting of ruthenium compounds and osmium compounds. In an embodiment, co-catalysts may be one or more ruthenium compounds. In an embodiment, the co-catalysts may be one or more acetates.

The reaction rate depends upon the concentration of the catalyst in the reaction mixture in reactor 110. In an embodiment, the catalyst concentration may be in a range from about 1.0 mmol to about 100 mmol catalyst per liter (mmol/1) of reaction mixture. In some embodiments the catalyst concentration is at least 2.0 mmol/l, or at least 5.0 mmol/l, or at least 7.5 mmol/l. In some embodiments the catalyst concentration is at most 75 mmol/l, or at most 50 mmol/l, or at least 25 mmol/l. In particular embodiments, the catalyst concentration is from about 2.0 to about 75 mmol/l, or from about 5.0 to about 50 mmol/l, or from about 7.5 to about 25 mmol/l.

In an embodiment, the carbonylation reaction in reactor 110 of system 100 may be performed in the presence of a catalyst stabilizer. Suitable catalyst stabilizers include at least two types of catalyst stabilizers. The first type of catalyst stabilizer may be a metal iodide salt such as lithium iodide. The second type of catalyst stabilizer may be a non-salt stabilizer. In an embodiment, non-salt stabilizers may be pentavalent Group VA oxides, such as that disclosed in U.S. Pat. No. 9,790,159 which is herein incorporated by reference. In an embodiment, the catalyst stabilizer may be one or more phosphine oxides. In an embodiment, the catalyst stabilizer may be CYTOP 503 from Solvay, previously sold under the tradename of Cyanex 923 from Cytec Corporation

The one or more phosphine oxides, in one or more embodiments, are represented by the formula R₃PO, where R is alkyl or aryl, O is oxygen, P is phosphorous. In one or more embodiments, the one or more phosphine oxides include a compound mixture of at least four phosphine oxides, where each phosphine oxide has the formula OPX₃, wherein O is oxygen, P is phosphorous and X is independently selected from C₄-C₁₈ alkyls, C₄-C₁₈ aryls, C₄-C₁₈ cyclic alkyls, C₄-C₁₈ cyclic aryls and combinations thereof. Each phosphine oxide has at least 15, or at least 18 total carbon atoms.

Examples of suitable phosphine oxides for use in the compound mixture include, but are not limited to, tri-n-hexylphosphine oxide (THPO), tri-n-octylphosphine oxide (TOPO), tris(2,4,4-trimethylpentyl)-phosphine oxide, tricyclohexylphosphine oxide, tri-n-dodecylphosphine oxide, tri-n-octadecylphosphine oxide, tris(2-ethylhexyl)phosphine oxide, di-n-octylethylphosphine oxide, di-n-hexylisobutylphosphine oxide, octyldiisobutylphosphine oxide, tribenzylphosphine oxide, di-n-hexylbenzylphosphine oxide, di-n-octylbenzylphosphine oxide, 9-octyl-9-phosphabicyclo [3.3.1]nonane-9-oxide, dihexylmonooctylphosphine oxide, dioctylmonohexylphosphine oxide, dihexylmonodecylphosphine oxide, didecylmonohexylphosphine oxide, dioctylmonodecylphosphine oxide, didecylmonooctylphosphine oxide, and dihexylmonobutylphosphine oxide and the like.

The compound mixture includes from 1 wt% to 60 wt%, or from 35 wt% to 50 wt% of each phosphine oxide based on the total weight of compound mixture. In one or more specific, non-limiting embodiments, the compound mixture includes TOPO, THPO, dihexylmonooctylphosphine oxide and dioctylmonohexylphosphine oxide. For example, the compound mixture may include from 40 wt% to 44 wt% dioctylmonohexylphosphine oxide, from 28 wt% to 32 wt% dihexylmonooctylphosphine oxide, from 8 wt% to 16 wt% THPO and from 12 wt% to 16 wt% TOPO, for example.

In one or more embodiments, the compound mixture exhibits a melting point of less than 20° C., or less than 10° C., or less than 0° C., for example.

The amount of pentavalent Group VA oxide, when used, is such that a ratio to rhodium is greater than about 60:1. In some embodiments, the ratio of the pentavalent Group 15 oxide to rhodium is from about 60:1 to about 500:1. In some embodiments, from about 0.1 to about 3 M of the pentavalent Group 15 oxide may be in the reaction mixture. In some embodiments, from about 0.15 to about 1.5 M, or from 0.25 to 1.2 M, of the pentavalent Group 15 oxide may be in the reaction mixture.

In other embodiments, the reaction may occur in the absence of a stabilizer selected from the group of metal iodide salts and non-metal stabilizers such as pentavalent Group 15 oxides. In further embodiments, the catalyst stabilizer may consist of a complexing agent which is brought into contact with the reaction mixture stream 111 in the flash vessel 120.

In an embodiment, hydrogen may also be fed into the reactor 110. Addition of hydrogen can enhance the carbonylation efficiency. In an embodiment, the concentration of hydrogen may be in a range of from about 0.1 mol % to about 5 mol % of carbon monoxide in the reactor 110. In an embodiment, the concentration of hydrogen may be in a range of from about 0.3 mol % to about 3 mol % of carbon monoxide in the reactor 110.

In an embodiment, the carbonylation reaction in reactor 110 of system 100 may be performed in the presence of water. In an embodiment, the concentration of water is from about 2 wt% to about 14 wt% based on the total weight of the reaction mixture. In an embodiment, the water concentration is from about 2 wt% to about 10 wt%. In an embodiment, the water concentration is from about 4 wt% to about 8 wt%.

In an embodiment, the carbonylation reaction may be performed in the presence of methyl acetate. Methyl acetate may be formed in situ. In embodiments, methyl acetate may be added as a starting material to the reaction mixture. In an embodiment, the concentration of methyl acetate may be from about 2 wt% to about 20 wt% based on the total weight of the reaction mixture. In an embodiment, the concentration of methyl acetate may be from about 2 wt% to about 16 wt%. In an embodiment, the concentration of methyl acetate may be from about 2 wt% to about 8 wt%. Alternatively, methyl acetate or a mixture of methyl acetate and methanol from byproduct streams of the methanolysis of polyvinyl acetate or ethylene-vinyl acetate copolymers can be used for the carbonylation reaction.

In an embodiment, the carbonylation reaction may be performed in the presence of methyl iodide. Methyl iodide may be a catalyst promoter. In an embodiment, the concentration of MeI may be from about 0.6 wt% to about 36 wt% based on the total weight of the reaction mixture. In an embodiment, the concentration of MeI may be from about 4 wt% to about 24 wt%. In an embodiment, the concentration of MeI may be from about 6 wt% to about 20 wt%. Alternatively, MeI may be generated in the reactor 110 by adding HI.

In an embodiment, methanol and carbon monoxide may be fed to the reactor 110 in stream 112 and stream 114, respectively. The methanol feed stream to the reactor 110 may come from a syngas-methanol facility or any other source. Methanol does not react directly with carbon monoxide to form acetic acid. It is converted to MeI by the HI present in the reactor 110 and then reacts with carbon monoxide and water to give acetic acid and regenerate the HI.

In an embodiment, the carbonylation reaction in reactor 110 of system 100 may occur at a temperature within the range of about 120° C. to about 250° C., alternatively, about 150° C. to about 250° C., alternatively, about 150° C. to about 200° C. In an embodiment, the carbonylation reaction in reactor 110 of system 100 may be performed under a pressure within the range of about 200 psia (14 kg/cm²) to 2000 psia (140 kg/cm²), alternatively, about 200 psia (14 kg/cm²) to about 1,000 psia (70 kg/cm²), alternatively, about 300 psia (21 kg/cm²) to about 500 psia (35 kg/cm²).

In an embodiment, the reaction mixture may be withdrawn from the reactor 110 through stream 111 and is flashed in flash vessel 120 to form a vapor stream 126 and a liquid stream 121. The reaction mixture in stream 111 may include acetic acid, methanol, methyl acetate, methyl iodide, carbon monoxide, carbon dioxide, water, HI, heavy impurities, catalyst, or combinations thereof. The flash vessel 120 may comprise any configuration for separating vapor and liquid components via a reduction in pressure. For example, the flash vessel 120 may comprise a flash tank, nozzle, valve, or combinations thereof.

The flash vessel 120 may have a pressure below that of the reactor 110. In an embodiment, the flash vessel 120 may have a pressure of from about 10 psig (0.7 kg/cm²) to 100 psig (7 kg/cm²). In an embodiment, the flash vessel 120 may have a temperature of from about 100° C. to 160° C.

The vapor stream 126 may include acetic acid and other volatile components such as methanol, methyl acetate, methyl iodide, carbon monoxide, carbon dioxide, water, entrained HI, complexed HI, and mixtures thereof. The liquid stream 121 may include the catalyst, complexed HI, HI, an azeotrope of HI and water, and mixtures thereof. The liquid stream 121 may further comprise sufficient amounts of water and acetic acid to carry and stabilize the catalyst, non-volatile catalyst stabilizers, or combinations thereof. The liquid stream 121 may recycle to the reactor 110. The vapor stream 126 may be communicated to light-ends column 130 for distillation.

In an embodiment, the vapor stream 126 may be distilled in a light-ends column 130 to form an overhead stream 132, a crude acetic acid product stream 136, and a bottom stream 131. In an embodiment, the light-ends column 130 may have at least 10 theoretical stages or 16 actual stages. In an alternative embodiment, the light-ends column 130 may have at least 14 theoretical stages. In an alternative embodiment, the light-ends column 130 may have at least 18 theoretical stages. In embodiments, one actual stage may equal approximately 0.6 theoretical stages. Actual stages can be trays or packing. The reaction mixture may be fed via stream 126 to the light-ends column 130 at the bottom or the first stage of the column 130.

Stream 132 may include HAc, water, carbon monoxide, carbon dioxide, methyl iodide, methyl acetate, methanol and acetic acid, and mixtures thereof. Stream 131 may have acetic acid, methyl iodide, methyl acetate, HI, water, and mixtures thereof. Stream 136 may have acetic acid, HI, water, heavy impurities, and mixtures thereof.

In an embodiment, the light-ends column 130 may be operated at an overhead pressure within the range of 20 psia (1.4 kg/cm²) to 40 psia (2.8 kg/cm²), alternatively, the overhead pressure may be within the range of 30 psia (2 kg/cm²) to 35 psia (2.5 kg/cm²). In an embodiment, the overhead temperature may be within the range of 95° C. to 135° C., alternatively, the overhead temperature may be within the range of 110° C. to 135° C., alternatively, the overhead temperature may be within the range of 125° C. to 135° C. In an embodiment, the light-ends column 130 may be operated at a bottom pressure within the range of 25 psia (1.8 kg/cm²) to 45 psia (3.2 kg/cm²), alternatively, the bottom pressure may be within the range of 30 psia (2.1 kg/cm²) to 40 psia (2.8 kg/cm²).

In an embodiment, the bottom temperature may be within the range of 115° C. to 155° C., alternatively, the bottom temperature is within the range of 125° C. to 135° C. In an embodiment, crude acetic acid in stream 136 may be emitted from the light-ends column 140 as a liquid sidedraw. Stream 136 may be operated at a pressure within the range of 25 psia (1.8 kg/cm²) to 45 psia (3.2 kg/cm²), alternatively, the pressure may be within the range of 30 psia (2.1 kg/cm²) to 40 psia (2.8 kg/cm²). In an embodiment, the temperature of stream 136 may be within the range of 110° C. to 140° C., alternatively, the temperature may be within the range of 125° C. to 135° C. Stream 136 may be taken between the fifth to the eighth actual stage of the light-ends column 130.

In one or more embodiments, the crude acetic acid in stream 136 may be optionally subjected to further purification, such as, but not limited to, drying-distillation, in drying column 140 to remove water and heavy-ends distillation in stream 141. Stream 141 may be communicated to heavy-ends column 150 where heavy impurities such as propionic acid may be removed in stream 151 and final acetic acid product may be recovered in stream 156.

The overhead stream 132 from the light-ends column 130 may be condensed and separated in a decanter 134 to form a light aqueous phase and a heavy organic phase. A portion or all of the heavy organic phase may be sent as stream 138 for further processing, as discussed below. Further, a portion of stream 138 may be optionally recycled to the reactor 110 via stream 112, for example. It should be noted that the portion of stream 138 sent for further processing (FIG. 1A) and the other portion of the stream 138 recycled to the reactor 110 may each originate as independent streams from the decanter 134 heavy organic phase. The light aqueous phase from the decanter 134 may be recycled to the light-ends column 130 in stream 135 or may be recycled to the reactor 110 in stream 139 via stream 112, for example.

The heavy organic phase stream 138 may have HAc, MeI, methyl acetate, hydrocarbons, acetic acid, water, and mixtures thereof. In an embodiment, stream 138 may be essentially non-aqueous with a water concentration of less than 1 wt%. In an embodiment, stream 138 may have MeI greater than 50% by weight of the stream. The light aqueous phase in streams 136 and 139 may have water (greater than 50% by weight of the stream), acetic acid, methyl acetate, methyl iodide and acetic acid, and mixtures thereof. Make-up water may be introduced into the decanter 134 via stream 133.

At least a portion of the heavy organic phase from the decanter 134 is sent via stream 138 to a distillation column, e.g., hydrocarbons removal column, alkanes column, etc. (depicted exemplary as 170 in FIG. 2A) to separate MeI from hydrocarbon (e.g., heavy hydrocarbons, alkanes). In one example of an alkanes column or alkanes tower, the stream 138 is distilled to form a vapor stream comprising the over 50% of the methyl iodide from the heavy organic phase 138 from the decanter 134 and a bottoms stream comprising over 50% of acetic acid, over 50% of methyl acetate, over 50% of methyl iodide, and over 50% of the hydrocarbon impurities from the heavy organic phase 138 from the decanter 134.

The overhead temperature of the distillation in the alkanes column is below about 75° C. so that the vapor stream is essentially free of the hydrocarbon impurities. In particular examples, the overhead temperature of the distillation is within the range of about 43° C. (boiling point of MeI) to about 75° C., about 43° C. to about 60° C., or about 43° C. to about 45° C. The closer the overhead temperature of the distillation to the boiling point of MeI, the less the amount of hydrocarbon impurities existing in the vapor stream. The vapor stream is recycled to the carbonylation reaction. Lowering the overhead temperature of the heavy organic phase distillation, although desirably reducing the hydrocarbon impurities in the vapor stream, results undesirably in a higher concentration of MeI in the bottoms stream. According to certain embodiments, the bottoms stream is disposed as a waste.

It should be noted that removal of the troublesome byproduct HAc from the acetic acid system 100 via physical or chemical techniques has occupied significant research time in the art for over a decade. This problematic byproduct and its aldehyde derivatives may unfortunately impact product purity. The HAc may also serve undesirably as a precursor to various hydrocarbons which impact decanter 134 heavy density, and as a precursor to higher alkyl iodides which may require expensive adsorption beds for their removal, for example.

As discussed below, the present techniques provide for a polymer-bound polyol resin pathway to remove a portion of the HAc from the from the acetic acid system 100 through adsorption onto the polymer-bound polymer resin. Resin concentration and conditions can be tailored to facilitate control of the rate of adsorption.

### Adsorption of HAc

According to the present techniques, HAc may be removed from the acetic acid system 100 by providing a stream comprising HAc from the acetic acid system 100 and contacting the stream (e.g., 138) with a polymer-bound polyol resin. Upon contacting the stream with the polymer-bound polyol resin, at least a portion of the HAc in the stream may be adsorbed onto the polymer-bound polyol resin.

At the outset, it should be noted that while the present disclosure focuses as an example on the treatment of stream 138 (decanter 134 heavy organic phase), other streams (comprising HAc) in the acetic acid system 100 may be treated polymer-bound polyol resin in accordance with the present techniques. For example, the stream 138 may be removed from the decanter 134 and at least a portion transferred to a distillation column (e.g., drying column 140, heavy-ends column 150, or combinations thereof), where an overhead stream comprising the solution (e.g., stream 142, stream 152, or combinations thereof) distilled from the heavy organic phase stream. That overhead stream may be contacted with the polymer-bound polyol resin according to the disclosed process, for instance.

As indicated, the byproduct HAc in the acetic acid process 100 may be difficult to remove from the process. There are few places in the system where HAc is sufficiently concentrated to efficiently target its removal. One location where HAc is sufficiently concentrated is the decanter 134 and in particular, the decanter 134 heavy organic phase (stream 138) where HAc is concentrated to about 0.5 wt%, for example. Physical removal of HAc from this heavy organic phase via distillation may be difficult as the HAc boiling point (20.2° C.) is close to that of the principal component in the heavy organic phase, MeI (boiling point=42.5° C.). Thus, the present techniques take advantage of HAc’s reactivity to adsorb HAc onto polymer-bound polyol to remove the adsorbed HAc form the acetic acid system 100.

Referring to FIG. 1A, and according to the present techniques, at least a portion of the heavy organic phase (stream 138) is contacted with a polymer-bound polyol resin, e.g., in a resin vessel 160. In an embodiment, about 5% to about 100% by weight of the heavy organic phase exiting the decanter 134 (i.e., in stream 138) is contacted with a polymer-bound polyol resin. In other embodiments, about 5% to about 50% by weight of the heavy-organic phase exiting the decanter 134 is contacted with a polymer-bound polyol resin. Portions of the remainder of the heavy-organic phase 138 exiting the decanter 134 may be recycled (see FIG. 1) to the reaction zone 102 and/or bypass (not shown) the resin vessel 160 (FIG. 1A) to the alkanes column 170, for example.

Returning to FIG. 1A, in embodiments, contacting the solution with the polymer-bound polyol resin (e.g., in resin vessel 160) may occur at room temperature, ambient temperature, or a temperature below the boiling point of HAc, and so on. In an embodiment, contacting the solution with the polymer-bound polyol resin may occur for at least 10 minutes, alternatively at least 15 minutes, alternatively at least 20 minutes, or alternatively at least 30 minutes. The mass ratio of aldehyde to polymer-bound polyol resin may be in a range of about 0.1 to about 2.0, for example.

Thus, in an embodiment, a stream 138 is discharged from the heavy organic phase of the decanter 134. This heavy organic phase includes a solution of HAc and methyl iodide. At least a portion of the heavy organic phase (stream 138) may pass to the resin vessel 160, where the solution may be contacted with the polymer-bound polyol resin according to the disclosed process. Moreover, it should be noted again that while certain embodiments focus on the decanter 134 heavy organic phase (stream 138) where acetaldehyde is present at about 0.5 wt%, for example, other streams comprising HAc (and MeI) in the acetic acid system 100 may be treated in accordance with the present techniques. In some embodiments, vessel 160 comprises a flow-through bed comprising the polymer-bound polyol.

In some embodiments, the polymer component of the polymer-bound polyol resin is selected from the group consisting of polystyrene crosslinked with divinylbenzene, polystyrene crosslinked with triphenylphosphine, or a combination thereof. In some embodiments, the polyol component of the polymer-bound polyol resin comprises polyols having two hydroxyl groups, three hydroxyl groups, or a combination thereof. In some embodiments, the polyol component of the polymer-bound polyols consists essentially of polymer-bound polyols derived from polyols having two hydroxyl groups, three hydroxyl groups, or a combination thereof. In some embodiments, the polyol component of the polymer-bound polyols comprises one or more diols. In some embodiments, the polyol component of the polymer-bound polyols consists essentially of one or more diols. In some embodiments, the polyol component of the polymer-bound polyol comprises is selected from the group consisting of 2-hydroxymethyl-1,3-propanediol, glycerol, or a combination thereof.

In some embodiments, the heavy organic phase stream 138 comprises methyl iodide, acetic acid, water, and a first concentration of acetaldehyde. When the heavy organic phase stream 138 is routed through the flow-through bed comprising the polymer-bound polyol resin, a portion of the acetaldehyde is adsorbed onto the resin such that the effluent from resin vessel 160 is a treated heavy organic phase stream 162 having a second concentration of acetaldehyde which is lower than the first concentration. In some embodiments, the ratio of the second concentration to the first concentration is less than or equal to 0.75, less than or equal to 0.65, or less than or equal to 0.55.

In some embodiments, the heavy organic phase stream 138 is contacted with the polymer-bound resin in resin vessel 160, and hence the absorption of a portion of the acetaldehyde in heavy organic phase stream 138, occurs at a temperature in the range of from 20° C. to 135° C., or 20° C. to 50° C.

In some embodiments, the heavy organic phase stream 138 is contacted with the polymer-bound resin in resin vessel 160, and hence the absorption of a portion of the acetaldehyde in heavy organic phase stream 138, occurs at a pressure in the range of from 14.7 psia (1.0 kg/cm²) to 263 psia (18.5 kg/cm²), or 14.7 psia (1.0 kg/cm²) to 40 psia (2.8 kg/cm²). In some embodiments, the pressure in resin vessel 160 is greater than or equal to the vapor pressure of HAc at the temperature in resin vessel 160.

In some embodiments, the heavy organic phase stream 138 is contacted with the polymer-bound resin in resin vessel 160, and hence the absorption of a portion of the acetaldehyde in the heavy organic phase stream 138, occurs when the in the heavy organic phase stream 138 is in contact with the polymer-bound resin in resin vessel 160 for at least about 10 minutes, 15 minutes, 20 minutes, or 30 minutes.

In some embodiments, the heavy organic phase stream 138 is contacted with the polymer-bound resin in resin vessel 160, and hence the absorption of a portion of the acetaldehyde in heavy organic phase stream 138, is performed in the absence of a strong acid or at least the absorption occurs when the reaction zone is free of or substantially free of a strong acid. As used herein, “strong acid” refers to an acid that completely ionizes in water, including, but not limited to, hydrochloric acid, hydrobromic acid, hydroiodic acid (“HI”), sulfuric acid, nitric acid, chloric acid, and perchloric acid. Strong acids can further include mineral acids, sulfonic acids (such as para-toluene sulfonic acid and methanesulfonic acid), heteropolyacids (such as tungstosilic acid, phosphotungstic acid and phosphomolybdic acid), and any of these acids when bound to a matrix (such as Amberlyst™ 15, which is a resin with bound sulfonic acid groups).

In some embodiments, the heavy organic phase stream 138 is contacted with the polymer-bound resin in resin vessel 160, and hence the absorption of a portion of the acetaldehyde in heavy organic phase stream 138, is performed under conditions wherein the mass ratio of acetaldehyde to polymer-bound polyol is in a range of from 0.1 to about 2.0, or 0.1 to 1.0.

FIG. 2 shows the effect of a polymer-bound diol on the acetaldehyde content in a static slurry of a polymer-bound polyol in a solution of an alkane and acetaldehyde at room temperature. The details of the experiment are shown in Example 1, below. FTIR analysis of the solution over time shows acetaldehyde content dropping by approximately 40%, which is believed to be the result of adsorption of the acetaldehyde onto the polymer-bound polyol.

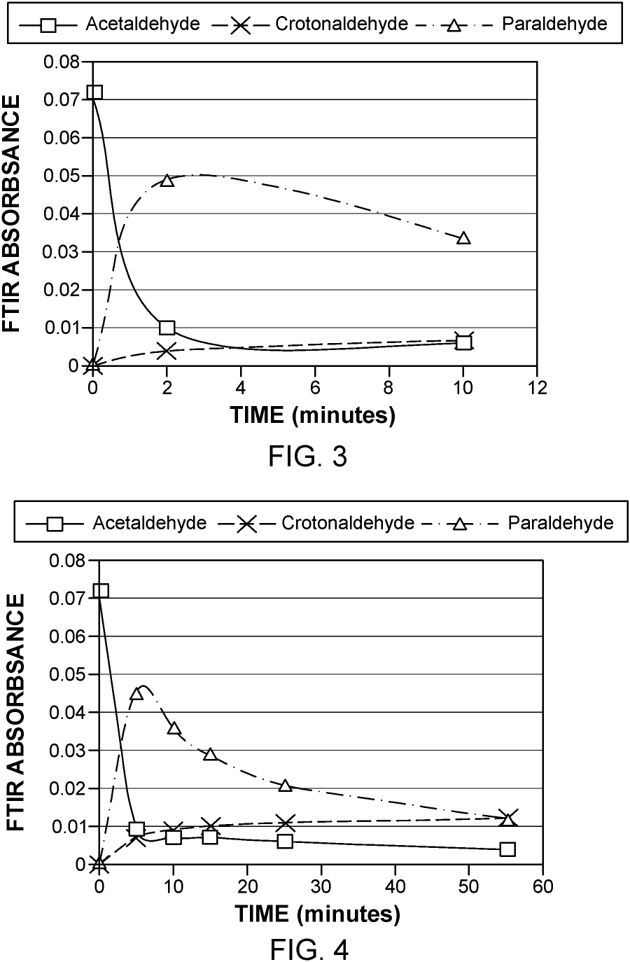

FIG. 3 shows the effect of a polymer-bound diol in combination with an acid catalyst on the acetaldehyde content in a static slurry of a polymer-bound polyol and an acid resin in a solution of an alkane and acetaldehyde at room temperature. The details of the experiment are shown in Example 2, below. FTIR analysis of the solution over time shows acetaldehyde content dropping but being replaced by the creation of paraldehyde and/or crotonaldehyde. It is believed that this shows that the catalytic transformation of acetaldehyde to paraldehyde and/or crotonaldehyde overtakes and/or suppresses the adsorption of acetaldehyde onto the polymer-bound resin.

FIG. 4 shows the effect of an acid catalyst alone on the acetaldehyde content in a static slurry of the acid resin in a solution of an alkane and acetaldehyde at room temperature. The details of the experiment are shown in Example 3, below. FTIR analysis of the solution over time shows acetaldehyde content dropping but being replaced by the creation of paraldehyde and/or crotonaldehyde, similar to the results shown in FIG. 3. It is believed that this reinforces the belief that the catalytic transformation of acetaldehyde to paraldehyde and/or crotonaldehyde overtakes and or suppresses the adsorption of acetaldehyde onto the polymer-bound resin, since the results shown in FIG. 3 and FIG. 4 show nearly the same results both with and without the polymer-bound polyol.

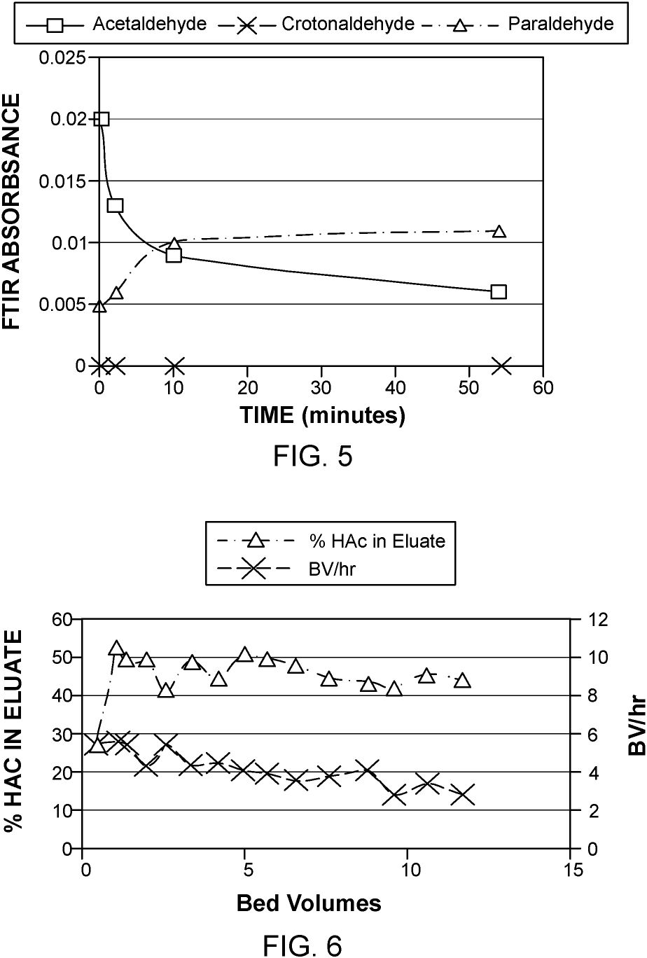

FIG. 5 shows the effect of a polymer-bound diol in combination with a small amount of a solution comprising a different acid catalyst on the acetaldehyde content in a static slurry of a polymer-bound polyol in a solution of an alkane, an acid, and acetaldehyde at room temperature. The details of the experiment are shown in Example 4, below. FTIR analysis of the solution over time shows acetaldehyde content dropping but being substantially replaced by the creation of paraldehyde. It is believed that this shows that the catalytic transformation of acetaldehyde to paraldehyde overtakes and/or suppresses the adsorption of acetaldehyde onto the polymer-bound resin, even when the acid is not strong enough to promote the transformation of paraldehyde to crotonaldehyde.

FIG. 6 shows the effect of a polymer-bound diol on the acetaldehyde content in a flow-through bed on a solution of an alkane and acetaldehyde at room temperature. The details of the experiment are shown in Example 5, below. FTIR analysis of the solution at different flow rates shows acetaldehyde content dropping by approximately 50%, which is believed to be the result of adsorption of the acetaldehyde onto the polymer-bound polyol.

Returning to FIGS. 1 and 1A, in some embodiments, the treated heavy organic phase 162 is recycled to the process in the reaction area 102, in some instances at a location where the treated heavy organic phase 162 is returned to the reactor 110. In some embodiments, the treated heavy organic phase 162 is passed to a distillation column 170 (e.g., called the alkanes tower) to produce a high-boiling bottoms stream 172 and an overhead stream 174 (low boilers, primarily MeI and HAc). In some of these embodiments, high-boiling bottoms stream 172 is recycled to the process in the reaction area 102, in some instances at a location where the high-boiling bottoms stream 172 is returned to the reactor 110, and overhead stream 174 is recycled to the process in the reaction area 102, in some instances at a location where the overhead stream 174 is returned to the flash vessel 120. In other embodiments, a portion of the bottoms stream is disposed of as waste.

In one or more embodiments, the disclosed process may be performed in a continuous format. For example, two resin beds or two resin vessels (e.g., two resin vessels 160) may be disposed in parallel, and while one is being regenerated, the other is in operation. On the other hand, the disclosed process may be performed in a batch format. The resin vessel 160 may be in continuous or batch operation and may include a tank of dimension and material suitable for production of acetic acid.

### Summary

In some aspects, methods for removing acetaldehyde from an acetic acid system are disclosed. In an embodiment, a method comprises obtaining from the acetic acid system a solution, comprising methyl iodide and a first concentration of acetaldehyde. The solution is then contacted with a polymer-bound polyol, wherein: the polymer-bound polyol adsorbs a portion of the acetaldehyde to produce a treated solution; the treated solution has a second concentration of acetaldehyde; and the second concentration is lower than the first concentration. In other embodiments, the method further comprises any one or any combination of the following:
(a) the polymer bound polyol is contained within a flow-through bed;
(b) the polymer bound polyol is a polymer-bound diol, and in some embodiments is 2-hydroxymethyl-1,3-propanediol, glycerol, or a combination thereof;
(c) the polymer component of the polymer-bound polyol is bound to polystyrene crosslinked with divinylbenzene, polystyrene crosslinked with triphenylphosphine, or a combination thereof;
(d) the ratio of the second concentration to the first concentration is less than or equal to 0.75, less than or equal to 0.65, or less than or equal to 0.55;
(e) the solution comprises less than 1 wt% water and no acid;
(f) the method further comprises recycling the treated solution within the acetic acid system;
(g) contacting the solution with the polymer-bound polyol occurs in the absence of a strong acid;
(h) contacting the solution with the polymer-bound polyol occurs:
   (1) at a temperature in the range of from 20° C. to 135° C., for at least about 10 minutes, and wherein the mass ratio of acetaldehyde to polymer-bound polyol is in a range of from 0.1 to about 2.0; or
   (2) at a temperature in the range of from 20° C. to 50° C., for at least about 15 minutes, and wherein the mass ratio of acetaldehyde to polymer-bound polyol is in a range of from 0.1 to about 1.0.
(i) the acetic acid system comprises a decanter, and the method further comprises removing a heavy organic phase stream comprising the solution from the decanter, and passing the heavy organic phase stream to a flow-through bed,
   wherein the flow-through bed comprises the polymer-bound polyol, and contacting the solution with the polymer-bound polyol is performed in the flow-through bed.

In some aspects, methods for producing acetic acid are disclosed. In an embodiment, a method for producing acetic acid comprises:
(a) flashing a reaction mixture discharged from an acetic acid production reactor into a vapor stream and a liquid stream, the vapor stream comprising acetic acid, water, methanol, methyl acetate, methyl iodide, and acetaldehyde;
(b) separating the vapor stream by distillation into: (1) a product side stream comprising acetic acid and water; (2) a bottoms stream; and (3) an overhead stream comprising methyl iodide, water, methyl acetate, acetic acid, and acetaldehyde;
(c) condensing the overhead stream into: (1) a light aqueous phase comprising water, acetic acid, and methyl acetate; and (2) a heavy organic phase comprising methyl iodide, acetic acid, water, and a first concentration of acetaldehyde; and
(d) contacting at least a portion of the heavy organic phase with a polymer-bound polyol to produce a treated heavy organic stream having a second concentration of acetaldehyde, wherein the second concentration is lower than the first concentration.

In other embodiments, the method for producing acetic acid further comprises any one or any combination of the following:
(a) the polymer bound polyol is contained within a flow-through bed;
(b) the polymer bound polyol is a polymer-bound diol, or is 2-hydroxymethyl-1,3-propanediol, glycerol, or a combination thereof;
(c) the polymer component of the polymer-bound polyol is bound to polystyrene crosslinked with divinylbenzene, polystyrene crosslinked with triphenylphosphine, or a combination thereof;
(d) the ratio of the second concentration to the first concentration is less than or equal to 0.75, less than or equal to 0.65, orless than or equal to 0.55.
(e) the solution comprises less than 1 wt% water, and no acid;
(f) the method further comprises recycling the treated solution within the acetic acid system or to the acetic acid production reactor;
(g) contacting the solution with the polymer-bound polyol occurs in the absence of a strong acid;
(h) contacting the solution with the polymer-bound polyol occurs at a temperature in the range of from 20° C. to 135° C., for at least 10 minutes, and at a mass ratio of acetaldehyde to polymer-bound polyol is in a range of from 0.1 to about 2.0, or at a temperature in the range of from 20° C. to 50° C., for at least 15 minutes, and at a mass ratio of acetaldehyde to polymer-bound polyol is in a range of from 0.1 to about 1.0;
(i) the acetic acid system comprises a decanter, the method further comprises removing a heavy organic phase stream comprising the solution from the decanter, and passing the heavy organic phase stream to a flow-through bed comprising the polymer-bound polyol, and wherein contacting the solution with the polymer-bound polyol is performed in the flow-through bed; and

In some aspects, acetic acid production systems are disclosed. In an embodiment, an acetic acid production system comprises:
(a) a reactor to react methanol and carbon monoxide in the presence of a carbonylation catalyst to form acetic acid;
(b) a flash vessel that receives a reaction mixture comprising the acetic acid from the reactor;
(c) a distillation column that receives a vapor stream from the flash vessel;
(d) a decanter that receives a condensed overhead stream from the distillation column; and
(e) a flow-through bed that receives a heavy organic phase from the decanter;
   wherein the heavy organic phase comprises methyl iodide, and acetaldehyde, and the flow-through bed comprises a polymer-bound polyol to adsorb acetaldehyde.

Although the disclosed process and system have been described in detail, it should be understood that various changes, substitutions and alterations can be made herein without departing from the spirit and scope of the invention as defined by the appended claims. Moreover, the scope of the present application is not intended to be limited to the particular embodiments of the processes, machines, compositions, means, methods, and/or steps described in the specification. As one of the ordinary skill in the art will readily appreciate from the present disclosure, processes, machines, compositions, means, methods, and/or steps, presently existing or later to be developed that perform substantially the same function or achieve substantially the same result as the corresponding embodiments described herein, may be utilized according to the present invention. Accordingly, the appended claims are intended to include within their scope such processes, machines, compositions, means, methods, and/or steps.

### EXAMPLES

The following investigations and examples are intended to be illustrative only, and are not intended to be, nor should they be construed as, limiting the scope of the present invention in any way.

### Test Methods

In Examples 1-6, infrared spectra were collected on a Nicolet 6700 FTIR spectrometer obtained from Thermo Scientific. The spectrometer was equipped with a Smart Miracle accessory also obtained from Thermo Scientific. The accessory contained a 3 bounce, zinc selenide ATR crystal. Those skilled in the art of infrared spectroscopy will realize that use of such an accessory will allow infrared absorbance peaks of HAc (1733 cm⁻¹), crotonaldehyde (1702 cm⁻¹), and paraldehyde (1342 cm⁻¹ and 856 cm⁻¹), to be monitored and quantified. Examples 1-5 address static slurries or mixtures. Example 6 addresses a flow-through bed mode. FTIR absorbance values were converted to molar values based on standards in the 0-1 M range prepared separately for each of acetaldehyde, crotonaldehyde and paraldehyde in decane.

### Raw Materials

Raw materials used herein are shown in Table 1, below. Polymer backbone in all cases is polystyrene crosslinked with divinylbenzene or polystyrene crosslinked with polystyrene triphenylphosphine. All starting materials are available from Sigma Aldrich, St. Louis, Missouri.

TABLE 1

| | | |
|---|---|---|
| Label | Composition*** | Type/Grade |
| PSDVB | Polystyrene crosslinked with divinylbenzene | 200-400 mesh particle size, 2% crosslinked with divinylbenzene |
| PSTPP | Polystyrene crosslinked with polystyrene triphenylphosphine | 100-200 mesh particle size, 3.0-3.6 mmol triphenylphosphine per gram |
| PBP1* | Polymer-bound polyol-1 (PSDVB-2-hydroxymethyl-1,3 -propanediol) | 50-100 mesh, 1.5-2.0 mmol diol/g, 1% crosslinked with divinylbenzene |
| PBP2* | Polymer-bound polyol-2 (PSTPP-glycerol) | 1-2 mmol glycerol/g, 1% cross linked with divinylbenzene |
| A15 | Amberlyst™ 15 Dry. | 200-400 um particle size, < 1.6% moisture |

* These polymer-bound diols are available commercially with 1-3 mmol/g loadings.

### Static Slurry Experiments

### Example 1

A mixture of 0.497 g PBP1 and 3 ml of the HAc solution (0.67 M concentration in decane) was added to a vial and then slurried and stirred at 22° C. and periodically sampled. The vials were septum sealed in order to prevent volatilization of acetaldehyde and stirred in order to optimize contact between solution and PBP1. Sampling was carried out via needle and syringe. The data reported in TABLE 2 and depicted graphically in FIG. 2 shows the time profile for reaction with PBP1. About 40% of acetaldehyde disappears from solution in about 10 minutes as determined by FTIR measurement. No new peaks appear. In particular, there is no evidence of formation of either crotonaldehyde (“CA”) or paraldehyde (“PLD”). This was a surprising finding as no acid was present to catalyze the acetal formation process.

TABLE 2

| Time (min.) | FTIR Absorbance | | |
|---|---|---|---|
| | HAc | CA | PLD |
| 0 | 0.072 | 0 | 0 |
| 5 | 0.056 | 0 | 0 |
| 10 | 0.047 | 0 | 0 |
| 15 | 0.043 | 0 | 0 |
| 25 | 0.043 | 0 | 0 |
| 44 | 0.042 | 0 | 0 |

### Example 2

Example 1 was repeated with the exception that a mixture of 0.51 g of PBP1 and 0.48 g of A15 was added to 3 ml of the HAc solution (0.67 M concentration). The data reported in TABLE 3 and depicted graphically in FIG. 3 show that the acid catalyst, A15, dominates the reaction (see FIG. 3). In order to reflect a mass balance of HAc units, FTIR measurements were converted to a molar basis for CA and PLD. Since 2 moles of HAc are required for self-aldol condensation to 1 mole of CA, CA moles were multiplied by 2 to determine a HAc molar equivalent value. Since PLD is a trimer of HAc, PLD moles were multiplied by 3 to determine a HAc molar equivalent value. Calculated values are shown in TABLE 3.

Based on previously observed behavior of A15, as described in U.S. Pat. No. 8,969,613, fully incorporated by reference herein, the mass balance in TABLE 3 is believed to show that all HAc removal can be attributed to acid catalyzed formation of CA by the A15 resin without participation of PBP1. HAc was rapidly trimerized to PLD, followed by slow formation of CA crotonaldehyde and adsorption of a portion of the formed crotonaldehyde onto the A15.

TABLE 3

| Time (min.) | FTIR Absorbance | | | HAc Molar Equivalent | | | | HAc Mat. Bal. (%) |
|---|---|---|---|---|---|---|---|---|
| | HAc | CA | PLD | HAc | CA | PLD | Total | |
| 0 | 0.072 | 0 | 0 | 0.67 | 0 | 0 | 0.67 | 100 |
| 2 | 0.01 | 0.004 | 0.049 | 0.09 | 0.04 | 0.15 | 0.62 | 93 |
| 10 | 0.006 | 0.007 | 0.034 | 0.06 | 0.07 | 0.11 | 0.53 | 79 |

### Example 3

Example 1 was repeated with the exception that 0.494 g of A15 was added to 3 ml of the HAc solution (0.67 M concentration). The data are reported in TABLE 4 and depicted graphically in FIG. 4. FTIR values were converted to HAc molar equivalent as described in Example 2. It is clear that all HAc reduction in this instance result from acid-catalyzed PLD formation followed by aldol condensation to form CA and adsorption of a portion of the formed CA onto the A15.

TABLE 3 shows an accounting for 79% of the HAc units at 10 minutes, while TABLE 4 shows an accounting for 85% of the HAc units at 10 minutes. The HAc discrepancy between TABLES 3 and 4 at 10 minutes are believed to be within the limits of experimental error and support that PBP1 does not participate in HAc removal in the presence of a strong acid.

TABLE 4

| Time (min.) | FTIR Absorbance | | | HAc Molar Equivalent | | | | HAc Mat. Bal. (%) |
|---|---|---|---|---|---|---|---|---|
| | HAc | CA | PLD | HAc | CA | PLD | Total | |
| 0 | 0.072 | 0 | 0 | 0.67 | 0 | 0 | 0.67 | 100 |
| 5 | 0.009 | 0.007 | 0.045 | 0.08 | 0.14 | 0.42 | 0.64 | 96 |
| 10 | 0.007 | 0.009 | 0.036 | 0.06 | 0.18 | 0.33 | 0.57 | 85 |
| 15 | 0.007 | 0.01 | 0.029 | 0.06 | 0.2 | 0.27 | 0.53 | 79 |
| 25 | 0.006 | 0.011 | 0.021 | 0.05 | 0.22 | 0.21 | 0.48 | 72 |
| 55 | 0.004 | 0.012 | 0.012 | 0.04 | 0.22 | 0.12 | 0.38 | 57 |

### Example 4

Example 1 was repeated with the exception that a mixture of 0.32 g of PBP1 and 10 µl of HI was added to 3 ml of a HAc solution (0.23 M concentration) of 1.4 wt% in decane. The data are reported in TABLE 5 and depicted graphically in FIG. 5. FTIR values were converted to HAc molar equivalent as described in Example 2. The data shows that while the small amount of acid catalyst is sufficient to allow conversion of acetaldehyde to paraldehyde, the acid catalyst apparently does not provoke aldol condensation to crotonaldehyde. The data further show that the strong acid dominates the reaction with HAc, and again, PBP1 does not react with HAc in the presence of a strong acid.

TABLE 5

| Time (min.) | FTIR Absorbance | | | HAc Molar Equivalent | | | | HAc Mat. Bal. (%) |
|---|---|---|---|---|---|---|---|---|
| | HAc | CA | PLD | HAc | CA | PLD | Total | |
| 0 | 0.02 | 0 | 0.005 | 0.230 | 0.000 | 0.000 | 0.230 | 100 |
| 2 | 0.013 | 0 | 0.006 | 0.150 | 0.000 | 0.042 | 0.192 | 83 |
| 10 | 0.009 | 0 | 0.01 | 0.100 | 0.000 | 0.126 | 0.226 | 98 |
| 55 | 0.006 | 0 | 0.011 | 0.070 | 0.000 | 0.159 | 0.229 | 100 |

Examples 1-4 suggest that the PBP1 reaction with acetaldehyde only takes place in the absence of a strong acid catalyst, essentially the reverse of behavior observed in a homogeneous diol environment. Based on the percent acetaldehyde removals measured, calculated efficiencies are in the 90-100% range.

### Example 5

As a control experiment, a commercial sample of the parent resin, polystyrene crosslinked with divinylbenzene (“PSDVB”) was also investigated, as was a similar polystyrene crosslinked with triphenylphosphine (“PSTPP”) resin. All tests included 4 wt% HAc or crotonaldehyde in decane and 0.225 g HAc or crotonaldehyde/g resin. Aldehyde reduction was measured after 15 minutes in vial at room temperature. As the data in TABLE 6 shows, only those resins with bound diols show evidence for acetaldehyde removal. Based on the percent acetaldehyde removals measured, calculated efficiencies are in the 90-100% range. TABLE 6 also shows data for crotonaldehyde removal. These crotonaldehyde data were obtained in a similar fashion as described for acetaldehyde. The data show that these polymer-bound diols are not effective for any crotonaldehyde removal.

TABLE 6

| Resin | % HAc Removed | % CA Removed |
|---|---|---|
| PSDVB | 0 | 0 |
| PSTPP | 6 | 0 |
| PSDVB-Propanediol | 44 | 0 |
| PSTPP-Glycerol | 47 | 0 |

### Flow-Through Bed Experiment

### Example 6

One flow through bed experiment was performed. A solution of 1.4 wt% HAc in decane was passed through a bed having a bed volume (“BV”) of 3.2 ml (2 g) and a length-to-diameter ratio of 9:1. The bed contained 2 g PSDVB-Propanediol having 1-3 mmol propanediol/g resin. The data reported in TABLE 7 and depicted graphically in FIG. 6 in which about 50% removal efficiency was observed.

TABLE 7

| BV (no.) | Flow (ml/hr) | Flow (BV/hr) | % HAc in Eluate |
|---|---|---|---|
| 0.5 | 17.4 | 5.4 | 27.2 |
| 1.1 | 18.0 | 5.6 | 52.5 |
| 1.4 | 17.4 | 5.4 | 49.5 |
| 2 | 13.8 | 4.3 | 49.5 |
| 2.6 | 17.4 | 5.4 | 41.6 |
| 3.4 | 13.8 | 4.3 | 49.0 |
| 4.2 | 14.4 | 4.5 | 44.6 |
| 5 | 13.2 | 4.1 | 51.0 |
| 5.7 | 12.6 | 3.9 | 49.5 |
| 6.6 | 11.4 | 3.6 | 48.0 |
| 7.6 | 12.0 | 3.8 | 44.6 |
| 8.8 | 13.2 | 4.1 | 43.6 |
| 9.6 | 9.0 | 2.8 | 42.1 |
| 10.6 | 10.8 | 3.4 | 45.5 |
| 11.7 | 9.0 | 2.8 | 44.1 |

The particular embodiments disclosed above are illustrative only, as the process and system may be modified and practiced in different but equivalent manners apparent to those skilled in the art having the benefit of the teachings herein. In the event of conflict between one or more of the incorporated patents or publications and the present disclosure, the present specification, including definitions, controls. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. A method for removing acetaldehyde from an acetic acid system, comprising:
(a) obtaining from the acetic acid system a solution, comprising methyl iodide and a first concentration of acetaldehyde; and
(b) contacting the solution with a polymer-bound polyol, wherein the polymer-bound polyol adsorbs a portion of the acetaldehyde to produce a treated solution, the treated solution has a second concentration of acetaldehyde, and the second concentration is lower than the first concentration.

2. The method of claim 1, wherein the polymer bound polyol is contained within a flow-through bed.

3. The method of claim 1, wherein the polymer bound polyol is a polymer-bound diol.

4. The method of claim 3, wherein the polymer-bound diol is 2-hydroxymethyl-1,3-propanediol, glycerol, or a combination thereof.

5. The method of claim 1, wherein the polymer component of the polymer-bound polyol is selected from polystyrene crosslinked with divinylbenzene, polystyrene crosslinked with triphenylphosphine, or a combination thereof.

6. The method of claim 1, wherein the ratio of the second concentration to the first concentration is less than or equal to 0.75.

7. The method of claim 1, wherein the solution comprises less than 1 wt% water.

8. The method of claim 1, further comprising recycling the treated solution within the acetic acid system.

9. The method of claim 8, wherein the treated heavy organic stream is recycled to the acetic acid production reactor.

10. The method of claim 1, wherein contacting the solution with the polymer-bound polyol occurs in the absence of a strong acid.

11. The method of claim 1, wherein contacting the solution with the polymer-bound polyol occurs at a temperature in the range of from 20° C. to 135° C., for at least 10 minutes, and at a mass ratio of acetaldehyde to polymer-bound polyol is in a range of from 0.1 to about 2.0.

12. The method of claim 1, wherein the contacting is performed at a pressure in the range of from 14.7 psia (1.0 kg/cm²) to 263 psia (18.5 kg/cm²).

13. The method of claim 1, wherein contacting the solution with the polymer-bound polyol occurs at a mass ratio of acetaldehyde to polymer-bound polyol is in a range of from 0.1 to about 2.0.

14. The method of claim 1, wherein the acetic acid system comprises a decanter, the method further comprising:
(a) removing a heavy organic phase stream comprising the solution from the decanter; and
(b) passing the heavy organic phase stream to a flow-through bed;
wherein the flow-through bed comprises the polymer-bound polyol, and wherein contacting the solution with the polymer-bound polyol is performed in the flow-through bed.

15. A method for producing acetic acid, said method comprising:
(a) flashing a reaction mixture discharged from an acetic acid production reactor into a vapor stream and a liquid stream, the vapor stream comprising acetic acid, water, methanol, methyl acetate, methyl iodide, and acetaldehyde;
(b) separating the vapor stream by distillation into: (1) a product side stream comprising acetic acid and water; (2) a bottoms stream; and (3) an overhead stream comprising methyl iodide, water, methyl acetate, acetic acid, and acetaldehyde;
(c) condensing the overhead stream into: (1) a light aqueous phase comprising water, acetic acid, and methyl acetate; and (2) a heavy organic phase comprising methyl iodide, acetic acid, water, and a first concentration of acetaldehyde; and
(d) contacting at least a portion of the heavy organic phase with a polymer-bound polyol to produce a treated heavy organic stream having a second concentration of acetaldehyde, wherein the second concentration is lower than the first concentration.

16. The method of claim 15, wherein the polymer bound polyol is a polymer-bound diol.

17. The method of claim 15, wherein the polymer component of the polymer-bound polyol is selected from polystyrene crosslinked with divinylbenzene, polystyrene crosslinked with triphenylphosphine, or a combination thereof.

18. The method of claim 15, further comprising recycling the treated solution within the acetic acid system.

19. The method of claim 15, wherein contacting the solution with the polymer-bound polyol occurs in the absence of a strong acid.

20. An acetic acid production system comprising:
(a) a reactor to react methanol and carbon monoxide in the presence of a carbonylation catalyst to form acetic acid;
(b) a flash vessel that receives a reaction mixture comprising the acetic acid from the reactor;
(c) a distillation column that receives a vapor stream from the flash vessel;
(d) a decanter that receives a condensed overhead stream from the distillation column; and
(e) a flow-through bed that receives a heavy organic phase from the decanter; wherein:
the heavy organic phase comprises methyl iodide, and acetaldehyde; and
the flow-through bed comprises a polymer-bound polyol to adsorb acetaldehyde.
